(19) United States
(12) Patent Application Publication
(10) Pub. No.: US 2018/0243555 A1
(43) Pub. Date: Aug. 30, 2018

(54) SYSTEMS AND METHODS FOR TRANSCUTANEOUS DIRECT CURRENT BLOCK TO ALTER NERVE CONDUCTION

(71) Applicant: Case Western Reserve University, Cleveland, OH (US)

(72) Inventors: Kevin L. Kilgore, Cleveland, OH (US); Gustaf Van Acker, Cleveland, OH (US); Tina Vrabec, Cleveland, OH (US); Niloy Bhadra, Cleveland, OH (US); Narenda Bhadra, Cleveland, OH (US)

(73) Assignee: Case Western Reserve Univesity, Cleveland, OH (US)

(21) Appl. No.: 15/756,614

(22) PCT Filed: Sep. 8, 2016
(86) PCT No.: PCT/US2016/050643

§ 371 Date: Mar. 1, 2018

Related U.S. Application Data
(60) Provisional application No. 62/215,267, filed on Sep. 8, 2015.

(51) Int. Cl. A61N 1/20; A61N 1/04; A61N 1/36

(52) U.S. Cl. CPC A61N 1/205; A61N 1/0456; A61N 1/0492; A61N 1/0476; A61N 1/36014

(57) ABSTRACT

One aspect of the present disclosure relates a system that can alter (e.g., block or attenuate) conduction in a nerve by transcutaneous DC application (tDCB). The system can include a current generator that generates a DC. A first skin electrode can be coupled to the current generator to deliver the DC transcutaneously through a target nerve to a second skin electrode. Conduction in the target nerve is directly altered as a result of an electric field generated in response to the DC.

### RELATED APPLICATIONS

This application claims the benefit of U.S. Provisional Application No. 62/215,267, entitled “SYSTEMS AND METHODS FOR APPLYING CURRENT TRANSCUTANEOUSLY TO ALTER NERVE CONDUCTION,” filed Sep. 8, 2015. The entirety of this provisional application is hereby incorporated by reference for all purposes.

### GOVERNMENT FUNDING

This work was supported, at least in part, by grant number R01-NS-074149 from the National Institutes of Health (NIH)—National Institute of Neurological Disorders and Stroke (NINDS). The United States government may have certain rights in this invention.

### TECHNICAL FIELD

The present disclosure relates generally to altering nerve conduction and, more specifically, to systems and methods for transcutaneous application of direct current to alter nerve conduction.

### BACKGROUND

Neurological disorder can be characterized by undesirable neural activity, which can cause chronic side effects, like bladder dysfunction, autonomic dysfunction, pain, or spasmodic muscle contractions, which can worsen over time when left untreated. Traditional treatments to block this undesirable neural activity include pharmacological approaches or surgery; however, pharmaceuticals have a slow time course and may have undesirable side effects, and surgery is usually irreversible. Nerve conduction block using electrical stimulation is an expanding alternative treatment strategy to pharmacological approaches and surgery to down-regulate or blockade of such undesirable nerve activity. True electrical conduction block typically employs some variation of high-frequency alternating current in the kilohertz frequency range and/or direct current applied directly to the target nerve, requiring surgically implanted electrodes; however, the invasiveness of the surgically implanted electrodes limits the use of this electrical conduction block. Meanwhile, existing non-invasive electrical stimulation approaches activate, rather than block, local neural circuits, presumably providing inhibitory effects through indirect means, limiting efficacy of such non-invasive approaches.

### SUMMARY

The present disclosure relates generally to altering (e.g., down-regulating or blocking) nerve conduction. For example, the systems and methods described herein can be used to alter the uninhibited nerve conduction that causes bladder dysfunction, autonomic dysfunction, pain, and/or spasmodic muscle contractions in a patient affected with a neurological disorder. More specifically, the present disclosure relates to systems and methods for transcutaneous application of direct current (DC) to alter nerve conduction. For example, the DC can be applied transcutaneously between at least two surface electrodes geometrically configured on a patient's skin so as to direct the flow of the DC in a direction sufficient to alter the transmission of action potentials in a target nerve. Indeed, the transcutaneous application of DC can provide a direct block of nerve conduction at the site of delivery.

In one aspect, the present disclosure can include a method for altering conduction in a target nerve. The steps of the method can include: placing at least two electrodes on a surface of a patient's skin; applying a DC to a target nerve located between the at least two electrodes, wherein the DC has an amplitude sufficient to alter transmission of action potentials in the target nerve; and altering the transmission of the action potentials in the target nerve based on an electric field generated as a result of the applied DC.

In another aspect, the present disclosure can include a system that can alter conduction in a target nerve. The system can include a current generator that generates a DC. The current generator can be coupled to a first skin electrode that delivers the DC transcutaneously through a target nerve to a second skin electrode. Conduction in the target nerve can be altered as a result of an electric field generated in response to the DC.

In a further aspect, the present disclosure can include a method for altering conduction in a target nerve. A DC can be applied through a target nerve via a transcutaneous electrode pair. The DC can have amplitude sufficient to block or attenuate conduction in the target nerve. The transcutaneous electrode pair can be geometrically arranged on the surface of the patient's skin to direct the flow of the DC in a direction that facilitates generation of an electric field sufficient to block or attenuate the conduction in the target nerve.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic diagram showing a system that can alter conduction in a target nerve constructed in accordance with an aspect of the present disclosure;

FIG. 2 illustrates examples of direct current (DC) waveforms with anodic polarity (left) and cathodic polarity (right) that can be generated and applied by the system in FIG. 1;

FIG. 3 illustrates examples of biphasic DC waveforms that can be generated and applied by the system in FIG. 1;

FIGS. 4-6 are examples of DC waveforms that can be generated and applied by the system in FIG. 1;

FIG. 7 is a schematic illustration showing the system in FIG. 1 applied to a patient's skin (medial view);

FIG. 8 is an axial view of the system in FIG. 7;

FIG. 9 is a process flow diagram illustrating a method for using a transcutaneously-applied DC to alter conduction in a target nerve according to another aspect of the present disclosure;

FIG. 10 is a schematic illustration showing the experimental setup of a rat thigh. The rat sciatic nerve and branches were surgically exposed, and a proximal stimulating bipolar cuff electrode was placed around the proximal sciatic nerve, providing biphasic pulses at 1-2 Hz (A). These stimulus pulses provided maximal muscle twitch activation of either the gastrocnemius or the tibialis anterior, through their respective motor nerve branches. The alternate branches were severed to provide isolation of the target muscle. The resulting maximal muscle twitches were measured through a force transducer—in this figure in-line with ankle dorsiflexion (B). Midway through each trial, transcutaneous application of direct current to alter nerve conduction (tDCB) was applied over the intact nerve driving the muscle twitches (C), leading to attenuation of motor twitch force;

FIG. 11 is a schematic illustration showing an exemplary electrode orientation relative to a target nerve. A) Axial illustration of electrodes relative to leg, with nerve, muscle and skin indicated. Dashed lines indicate hypothetical electrical fields generated between cathodal and anodal electrodes placed in proximity to the target nerve. B) Mediolateral view with proximal stimulating electrode (PS) shown relative to the nerve, muscle and in-line force transducer (FT). Circled numbers indicate cathodal and anodal electrode pairings through which current was delivered. Compass rose indicates rat orientation in each view—ventral (V), dorsal (D), medial (M), lateral (L), rostral (R) and caudal (C);

FIG. 12 is a graph showing tDCB applied during maximal motor output (the force output resulting from proximal sciatic nerve stimulation at 2 Hz). This force is maximal when proximal stimulation only is applied, or during pre-DC baseline (black dashed horizontal line). When the DC field was applied (red solid tracing), motor output was decreased to a stable partial block of 94.8% during the plateau period (black solid line), or the period when DC was held at a constant level. Ramp up to, and ramp down from, the plateau was included to mitigate onset and offset motor activity associated with rapid changes in DC. Max force amplitude was the maximum force output achieved via proximal sciatic nerve stimulation, whereas 0 force amplitude was resting baseline with no stimulation;

FIG. 13 is a graph showing the relationship between direct current and conduction block. These data show the relationship between the amplitude of the direct current applied and the block percentage achieved, holding all other parameters constant. Solid red vertical lines are the force output driven by 2 Hz proximal stimulation, the attenuation of which midway through the trial was achieved with a DC current of 6 mA, indicated by the red ramping waveform. Colored triangles are the peak locations achieved with the associated color-coded DC waveforms. As the blocking current amplitude is increased, so too is the block percent achieved. Block at each current level was consistent for the ten-second duration of each tDCB plateau. Vertical dashed line indicates onset of ten-second DC plateau in which DC current was held constant. Max force amplitude was the maximum force output achieved via proximal sciatic nerve stimulation, whereas 0 force amplitude was resting baseline with no stimulation; and

FIG. 14 is a graph showing tDCB of tetanic muscle contraction. Tetanic activity of the tibialis anterior muscle was achieved by applying a 40 Hz biphasic stimulation train to the sciatic nerve proximally (PS Onset). tDCB was then applied (DC Waveform), resulting in partial block of the force output. Tetanic activity returned once tDCB was turned off, and tetanic activity ceased once the proximal stimulation was turned off (PS Offset). tDCB was applied at 20 mA. Max force amplitude was the maximum force output achieved via proximal sciatic nerve stimulation at 40 Hz, whereas 0 force amplitude was resting baseline with no stimulation.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “alter” or “altering”, when used with reference to nerve conduction, can refer to affecting or changing a manner in which action potentials are conducted in a target nerve. In some instances, nerve conduction can be altered by extinguishing an action potential at some point as it travels along the nerve (also referred to as “blocking” nerve conduction). In other instances, nerve conduction can be altered by increasing the activation threshold of a target nerve and/or decreasing the conduction velocity of a target nerve (also referred to as “attenuating” nerve conduction). Attenuating nerve conduction can lead to an incomplete block of nerve conduction to alter normal nerve activity (e.g., normal action potential conduction). In either case, when nerve conduction is altered, it can be directly blocked or attenuated.

As used herein, nerve conduction is “blocked” when transmission of action potentials through a target nerve is extinguished completely (e.g., 100% extinguished) as the action potentials travel through the nerve. The block can be achieved by depolarization or hyperpolarization of the nerve membrane comprising the target nerve. In other words, the term “blocked” can refer to a complete conduction block.

As used herein, nerve conduction is “attenuated” when an “incomplete nerve block” occurs. The term “incomplete block” can refer to a partial block, where less than 100% (e.g., less than about 90%, less than about 80%, less than about 70%, less than about 60%, or less than about 50%) of the action potentials traveling through a target nerve are extinguished. In one example, when nerve conduction is attenuated, the target nerve will have an increased activation threshold and thereby make the target nerve more difficult to excite. In other words, the term “attenuated” can refer to a stable partial conduction block.

Nerve conduction can be altered by applying an external electrical signal to a target nerve. For example, a “direct current” or “DC” can be applied to a target nerve so that an electric field generated by the DC is sufficient to alter the conduction in the target nerve. The DC can be of either polarity (e.g., either cathodic or anodic). In some instances, the DC can be applied as the first phase of a biphasic waveform. The second phase of the biphasic waveform can either reverse 100% of the total charge delivered by the first phase (as a charge-balanced biphasic waveform) or reverse less than 100% of the total charge delivered by the first phase, thereby reducing electrochemical reactions that are damaging to the skin surface and/or the electrodes used to deliver the DC.

A DC can be applied “transcutaneously” (e.g., through the skin) between at least two “surface electrodes” arranged about a target nerve. The surface electrodes can be made of a conductive material that is reversibly attachable to the surface of a patient's skin. In some instances, the surface electrodes can be attached to the surface of the patient's skin via a conductive gel that improves conduction of the DC through the skin.

As used herein, the term “nerve” can refer to one or more fibers that employ electrical and chemical signals to transmit motor, sensory, and/or autonomic information from one body part to another. A nerve can refer to either a component of the central nervous system or the peripheral nervous system.

As used herein, the term “reversible”, when referring to a nerve, can mean that the nerve is returning to normal conduction after an applied DC is removed from the nerve. In some instances, altered nerve conduction can be reversed in 120 seconds or less. In other instances, altered nerve conduction can be reversed in 60 seconds or less.

As used herein, the term “neurological disorder” can refer to a condition or disease characterized at least in part by abnormal conduction in one or more nerves. In some instances, a subject suffering from a neurological disorder can experience pain and/or muscle spasticity. Examples of neurological disorders can include stroke, brain injury, spinal cord injury (SCI), cerebral palsy (CP), multiple sclerosis (MS), etc.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

As used herein, the term “medical professional” can refer to an individual who provides care to a patient. A medical professional can be, for example, a doctor, a physician's assistant, a student, a nurse, a caregiver, or the like.

### II. Overview

The present disclosure relates generally to altering nerve conduction and, more specifically, to systems and methods for transcutaneous application of direct current to alter nerve conduction (hereinafter referred to as transcutaneous direct current block (tDCB)). Altering nerve conduction using tDCB can be used to treat an immense array of clinical ailments, providing a viable, inexpensive, non-pharmacological alternative treatment. Indeed, tDCB provides a completely non-invasive way to alter peripheral nerve transmission using electrodes placed on the skin surface, whose onset is immediate and action is readily reversible. Additionally, tDCB applies a constant current, leaving cells refractory to repeated depolarization. By doing so, tDCB can be purely inhibitory depending upon waveform characteristics.

The systems and methods described herein employing tDCB provide advantages over currently available non-invasive electrical stimulation-based approaches. Examples of these approaches include transcutaneous electrical nerve stimulation (TENS) and spinal cord stimulation (SCS). Specifically, TENS and SCS produce effects through indirect means, presumably by using an alternating current (AC) to activate either inhibitory or competitive circuits within the nervous system. The systems and methods of the present disclosure employ tDCB to operate under a fundamentally different principle. tDCB produces a direct block of nerve conduction at the delivery site. It is analogous to a local pharmacological blocking agent, except that it is produced electrically and has the unique capability of nearly instantaneous and reversible titrating and tapering for optimal efficacy.

Another method of transcutaneous electrical stimulation is called transcutaneous direct current stimulation (tDCS). tDCS involves applying DC for prolonged periods at low amplitudes, typically through the skull. tDCS produces an effect through stimulation of nerves, in contrast to tDCB. tDCS uses amplitudes insufficient to block, and delivers DC without charge balance for periods of 20-30 minutes at a time. The systems and methods of the present disclosure employ tDCB to operate under a fundamentally different principle. tDCB produces a direct block of nerve conduction at the delivery site.

DC has a good safety profile when controlling for parameters such as total charge, and has been used extensively for clinical and research applications in both transcranial and transspinal direct current stimulation. However transcutaneous DC has never been used to alter action potential conduction in peripheral nerve. DC can be applied transcutaneously to produce a DC field of sufficient strength and direction to block or otherwise inhibit or attenuate nerve conduction directly. The systems and methods of the present disclosure utilize current ranges that are significantly higher (>10 mA) than the maximum current used in tDCS (2 mA). This is accomplished by the systems and methods of the present disclosure by either limiting the total time of delivery below the level that causes damage to the skin, by using electrode materials and configurations that allow higher charge to be delivered through the skin, and/or by using a charge-balancing or charge-recovery phase that follows the active DC phase.

### III. Systems

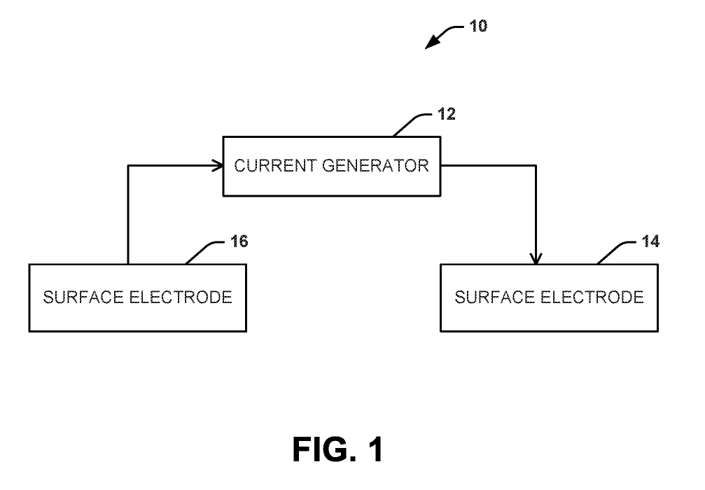

One aspect of the present disclosure can include a system 10 (FIG. 1) that can alter (e.g., block or attenuate) conduction in a target nerve by applying a direct current (DC) transcutaneously. The transcutaneous application of DC can be entirely non-invasive and can provide a direct attenuation of nerve conduction in a peripheral nerve (e.g., a motor nerve, a sensory nerve, and/or an autonomic nerve) at or near the site of delivery. The system 10 can produce a DC field of sufficient strength and direction to block or otherwise inhibit or attenuate nerve conduction. Advantageously, the system 10 can utilize a high current range by either limiting the total time of delivery below the level that causes damage to the skin, by using electrode materials and configurations that allow higher charge to be delivered through the skin, and/or by using a charge-balancing or charge-recovery phase that follows the active DC phase.

Generally, the system 10 can include components for generating a current (e.g., current generator 12) as well as components for transcutaneously applying the current (e.g., surface electrodes 14, 16) to alter conduction in a target nerve. Examples of target nerves can include peripheral nerves (e.g., motor, sensory, and/or autonomic nerves) and nerves or nervous tissue comprising the central nervous system (e.g., brain and/or spinal cord). As discussed in more detail below, transcutaneous application of DC to a target nerve can be used to treat various neurological disorders including, but not limited to, pain and muscle spasticity.

As shown in FIG. 1, the system 10 can include a current generator 12, a first surface electrode 14, and a second surface electrode 16. The first and second surface electrodes 14, 16 can be attached to a patient's skin and coupled to the current generator 12. In some instances, the first and second surface electrodes 14, 16 can be in electrical communication with the current generator 12 via a wired connection. In other instances, the first and second surface electrodes 14, 16 can be in electrical communication with the current generator 12 via a wireless connection and/or a combination of a wired connection and a wireless connection.

The first and second surface electrodes 14, 16 are configured as transcutaneous or skin electrodes, meaning that the electrodes can be applied to the surface of a patient's skin without penetrating the skin surface. The surface electrodes 14, 16 can be sized and dimensioned to facilitate delivery of current through the patient's skin to alter the conduction in the target nerve. For example, at least one of the surface electrodes 14, 16 can be shaped as a square, a rectangle, a circle, an oval, a triangle, or any other shape that can facilitate generation of the electric field under the patient's skin to alter the conduction in the target nerve. The surface electrodes 14, 16 can be made from at least one electrically-conductive material (e.g., stainless steel, platinum, gold, silver, carbon, carbon gel, conductive silicon rubber, conductive adhesive gel, or the like). In some instances, the surface electrodes 14, 16 can be constructed to be biocompatible so that the application to the patient's skin does not cause an unwanted reaction with the skin. In other instances, the surface electrodes 14, 16 can be coupled to the patient's skin by a gel or other protective substance. In some instances, the conductive gel or other electrolyte can create a large physical buffer to protect the skin from undesirable reaction products at the electrode-electrolyte interface. The gel can be a conductive gel (e.g., including an electrolyte) to improve conduction of the current through the patient's skin.

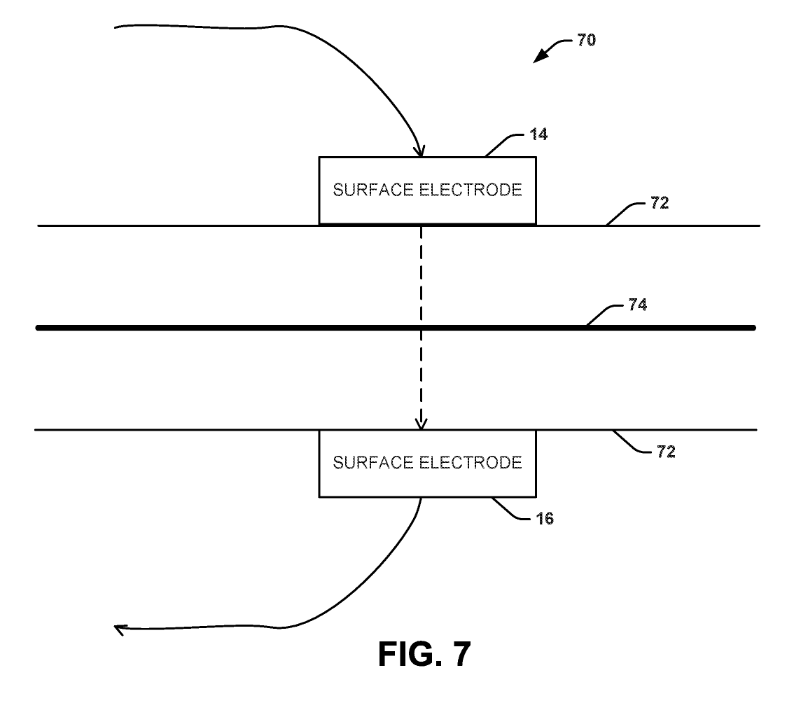

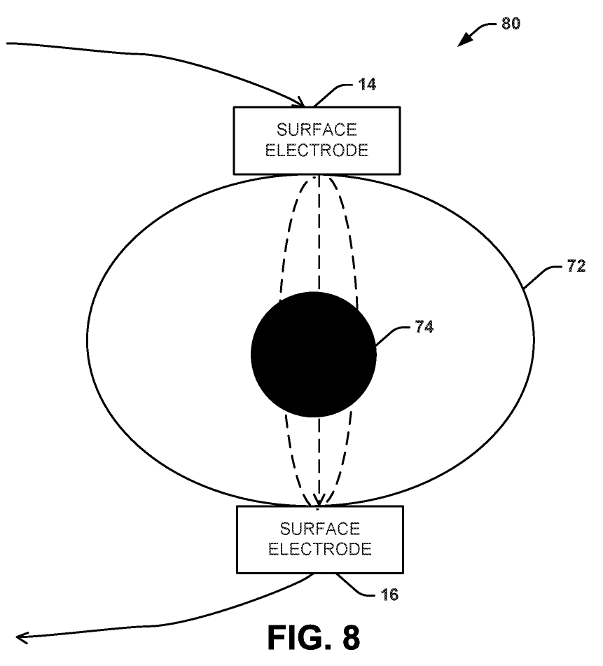

The surface electrodes 14, 16 can be geometrically arranged on the surface of the patient's skin to direct the flow of current in a direction sufficient to alter the transmission of action potentials in the target nerve. In one example, the at least two surface electrodes 14, 16 can be arranged on the patient's skin 72 on opposing sides of the target nerve 74, as shown in FIGS. 7-8. It will be appreciated that the system 10 can include a greater number of surface electrodes 14, 16 than those described herein. However, in most instances, a greater number of surface electrodes 14, 16 will still be geometrically arranged on the surface of the patient's skin to direct the flow of the current in a direction sufficient to alter the transmission of action potentials in the target nerve. It will be appreciated that additional electrodes can be used, for example, to shape the electric field generated by the DC.

The current generator 12 can be configured or programmed to generate a current, such as a DC. Accordingly, the current generator 12 can be any device configured or programmed to generate the specified current for transcutaneous application to a target nerve to achieve an alternation in conduction thereof. One example of a current generator 12 is a battery-powered, portable generator. Another example of a current generator 12 is an implantable generator (IPG). It will be appreciated that the current generator 12 can include additional components to configure the current waveform, such as an amplitude modulator (not shown).

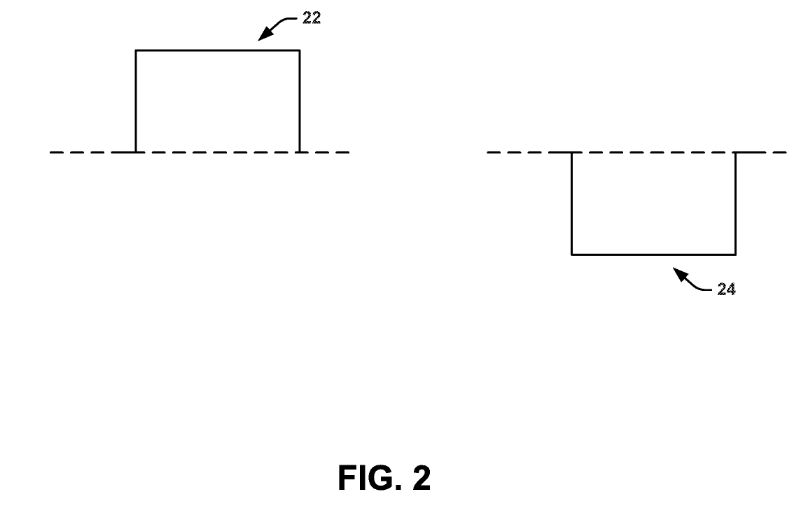

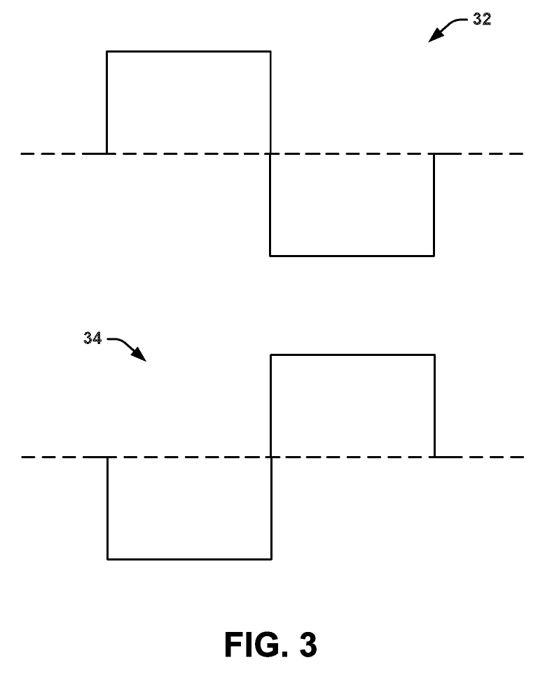

In some instances, the current generated by the current generator 12 can be a DC, as shown in FIG. 2. The generated DC can have an anodic polarity 22 or a cathodic polarity 24, and an amplitude sufficient to generate an electric field capable of altering conduction in a target nerve. The electric field can be a depolarizing or hyperpolarizing field that is generated within the patient's skin in proximity to the target nerve. In some instances, the current generator 12 can be configured or programmed to generate a DC having a biphasic waveform, as shown in FIG. 3. In this case, the altering DC can be delivered to the target nerve in the first phase for a specific period of time, while a second phase having an opposite polarity can reduce or eliminate unwanted effects (e.g., due to electrochemical reaction products) generated by the first phase. The unwanted effects can be generated and reversed at the surface electrodes 14, 16, at the skin, and/or at the electrode-skin interface.

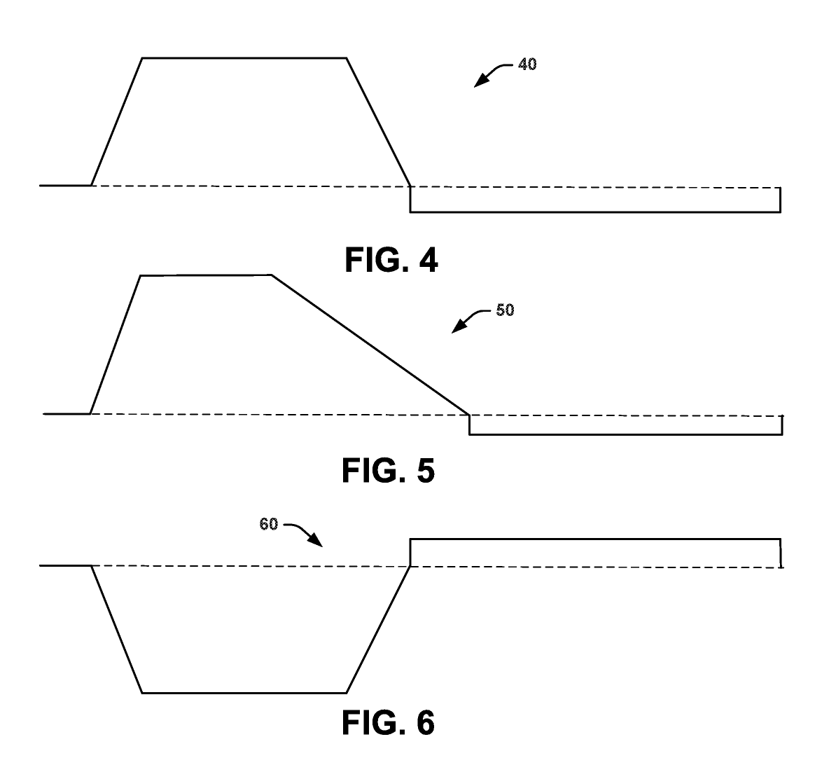

FIGS. 4-6 depict exemplary biphasic DC waveforms that can be generated by the current generator 12. In some instances, a generated biphasic DC waveform can be a charge-balanced biphasic waveform that produces zero net charge. In other instances, a generated biphasic DC waveform can be applied as a substantially charge-balanced DC waveform that produces a small net charge to reduce electrochemical reactions that are damaging to the skin surface and/or the surface electrodes 14, 16. Advantageously, the current generator 12 can be configured or programmed to a DC having a biphasic waveform, which allows nerve conduction to be altered without damaging the target nerve itself, the skin of the patient, and/or producing systemic side-effects. Additionally, the alteration induced by the delivered DC is reversible. For example, the target nerve can return to normal conduction in a short time period (e.g., within 60-120 seconds) after the application of the DC to the target nerve is terminated.

FIG. 7 is a schematic illustration showing the system in FIG. 1 applied to a patient's skin (medial view). FIG. 8 is an axial view of the system in FIG. 7. The DC (dashed arrow) can be applied by surface electrode 14 on the surface of the skin, through the patient's skin 72 and the target nerve 74, back out of the patient's skin 72 to surface electrode 16. The DC can establish a DC field (e.g., shown in FIG. 8) of sufficient strength and direction to block or otherwise inhibit or attenuate nerve conduction in the target nerve 74.

### IV. Methods

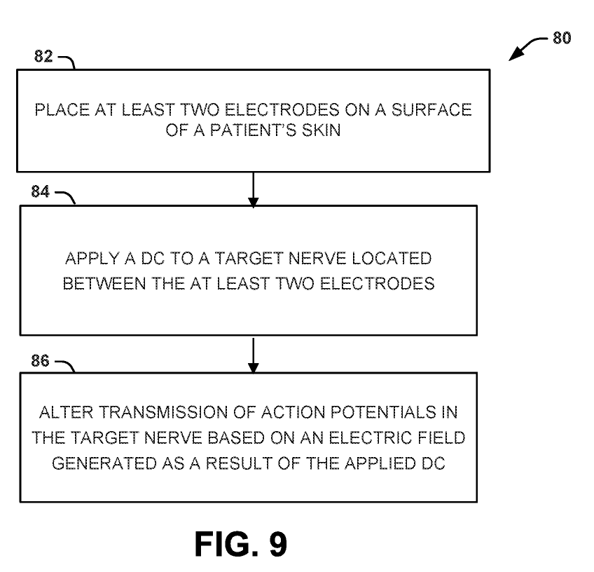

Another aspect of the present disclosure can include a method 80 (FIG. 9) for altering (e.g., blocking or attenuating) conduction in at least a portion of a target nerve by a transcutaneously applied current. The transcutaneous application is non-invasive, requiring no electrodes to be implanted within the patient's body. The method 80 can produce a direct current (DC) field of sufficient strength and direction to block or otherwise inhibit or attenuate nerve conduction. Advantageously, the method 80 can utilize a high current range by either limiting the total time of delivery below the level that causes damage to the skin, by using electrode materials and configurations that allow higher charge to be delivered through the skin, and/or by using a charge-balancing or charge-recovery phase that follows the active DC phase.

The method 80 can generally include the steps of: placing at least two electrodes on a surface of a patient's skin (Step 82); applying a DC to a target nerve located between the at least two electrodes (Step 84); and altering transmission of action potentials in the target nerve based on an electric field generated as a result of the applied DC (Step 86). The method 80 is illustrated as process flow diagrams with flowchart illustrations. For purposes of simplicity, the method 80 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 80.

At Step 82, at least two electrodes (e.g., surface electrodes 14, 16) can be placed on a surface of a patient's skin. The at least two electrodes can be sized and dimensioned to deliver an appropriate current to alter the conduction in at least a portion of a target nerve. The at least two electrodes can be geometrically arranged on the surface of the patient's skin to direct the flow of the DC in a direction sufficient to alter the transmission of action potentials in the target nerve. For example, the at least two electrodes can be arranged on opposing sides of the target nerve.

At Step 84, a current generator can be activated to generate a DC. The generated DC can be applied through the patient's skin so that conduction is altered in the target nerve located between the at least two electrodes. The applied DC can be anodic or cathodic and have an amplitude sufficient to generate an electric field able to alter transmission of action potentials in the target nerve. In some instances, DC can be applied as a biphasic waveform, such as one of those illustrated in FIGS. 4-6. The second phase of the biphasic waveform, in some instances, can reverse the charge delivered by the first phase. In other instances, the second phase can reverse less than 100% of the total charge delivered by a first phase of the biphasic waveform to reduce electrochemical reactions that are damaging to the skin surface and/or the electrodes.

At Step 86, transmission of the action potentials in the target nerve can be altered (e.g., blocked or attenuated) based on an electric field generated as a result of the applied DC. The transmission of the action potentials can be altered without damaging the structure of the target nerve, the skin of the patient, and/or producing systemic side-effects. Additionally, the altered nerve conduction is reversible such that conduction within the target nerve can return to normal in a short time period (e.g., within 60-120 seconds) after the application of the current ends.

### V. Examples

The following examples are for the purpose of illustration only is not intended to limit the scope of the appended claims.

### Example I—Rat In Vivo Experiments

This Example demonstrates the feasibility of altering conduction in a rat sciatic nerve via transcutaneous (surface) application of a DC (tDCB). In an in vivo rodent model, tDCB produced stable partial-to-complete neuromotor blockade of sciatic nerve branches, dependent on stimulus parameters and electrode geometry. Complete neuromotor block was achieved at tDCB amplitudes as low as 6 mA, and in 80% of subjects at or below 20 mA. Our results reveal that neuromotor activity can be rapidly, reliably and reversibly blocked using tDCB.

### Methods

### Surgical Procedure

Data were collected from 10 adult Sprague-Dawley rats during acute experiments. For each procedure general anesthesia was induced and maintained using isoflurane. Surgical exposure of the rat sciatic nerve and branches was performed as described previously. Briefly, the rat hindlimb was shaved and an incision was made superficial to the gluteus lengthwise and rostrocaudally, exposing the sciatic nerve from one centimeter lateral to midline to the tibial and peroneal nerve bifurcation. For preparations requiring tibialis anterior activation and block, the sural and tibial nerve branches were cut to eliminate conduction to the gastrocnemius, leaving conduction through the common peroneal nerve intact. In this preparation, force output of the tibialis anterior resulting in ankle dorsiflexion was measured using an in-line force transducer attached to the dorsum of the foot. For preparations requiring gastrocnemius activation and block, the sural and common peroneal nerves were severed, leaving the tibial nerve intact. The Achilles tendon was severed just proximal to the calcaneal insertion, and the proximal segment was attached to a force transducer via a clamp and suture. This attachment was tightened to a passive tension of approximately 1-2 N.

A platinum bipolar J-cuff electrode was placed circumferentially to encompass approximately 270° of the exposed sciatic nerve proximally. With the proximal stimulating electrode in place, muscle and skin were sutured closed. At the end of the experiment, the rat was humanely euthanized. All procedures were approved by the Institutional Animal Care and Use Committee and conformed to the Guide for the Care and Use of Laboratory Animals, published by the United States Department of Health and Human Services and the National Institutes of Health.

### Electrical Stimulation

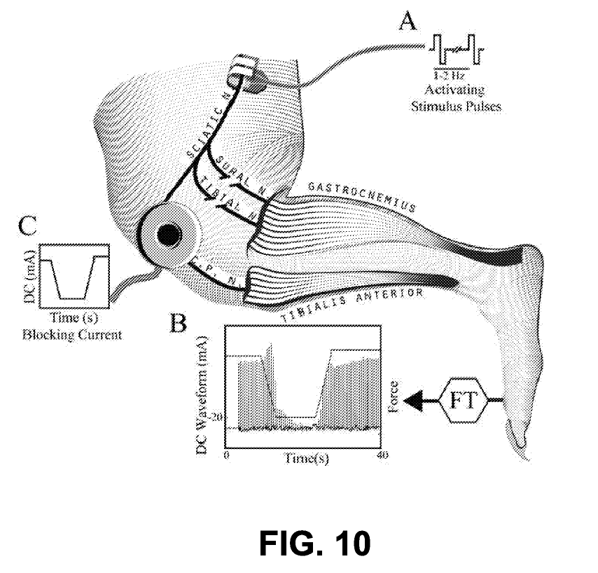

To evoke detectable muscle twitches via the distal force transducer, biphasic stimulation was delivered to the proximal end of the sciatic nerve using a platinum J-cuff electrode (FIG. 10A). The electrode was comprised of two exposed windows of platinum, each 2×1 mm in dimension, embedded in a silicone sheet and shaped in a J form in order to encompass approximately 270° of the exposed sciatic nerve. Cathode-leading biphasic stimulus pulses, 20 μs per phase, were applied to the proximal sciatic nerve at 1-2 Hz frequency. Saturation threshold of this signal for producing maximal muscle response was determined by monitoring the force output (FIG. 10B) while incrementally increasing the stimulus current until the force output plateaued, signifying maximal activation of the sciatic nerve. Once saturation threshold of this proximal stimulation was achieved, these stimulation parameters were applied concurrent with the blocking stimulation (FIG. 10C).

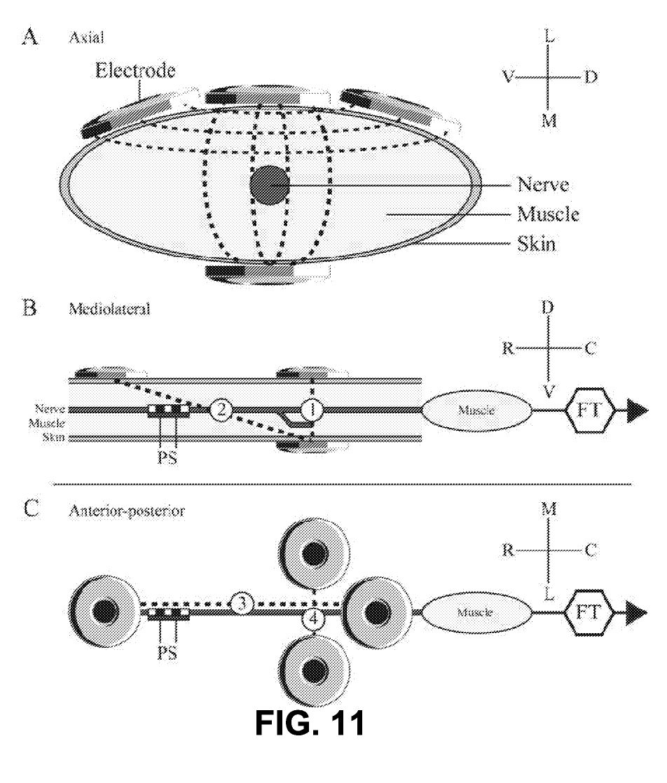

Blocking stimulation was applied transcutaneously to branches of the sciatic nerve distal to the implanted proximal stimulating electrode. Transcutaneous direct current block (tDCB) was applied through Ag/AgCl ring electrodes with an inner and outer diameter of 0.6 and 1.2 cm, respectively, and with a total surface area of approximately 0.85 cm² (EL-TP-RNG Sintered; Stens Biofeedback Inc, San Rafael, Calif.). Conductive gel (Signa, Parker Laboratories Inc., Fairfield, N.J., USA) was placed in a thin layer between the electrodes and skin surface. Active and reference electrodes were tested using multiple orientations relative to the target nerve (FIG. 11). Four general electrode configurations were investigated: active and return electrodes placed on opposing sides of the leg/nerve, oriented perpendicular to the target nerve (FIG. 11B-1); active and return electrodes placed on opposing sides of the leg/nerve, at acute or obtuse angles relative to the target nerve (FIG. 11B-2); active and return electrodes on the same side relative to the leg/nerve, oriented parallel to, and directly overlying, the target nerve (FIG. 11C-3); and active and return electrodes on the same side with respect to the leg/nerve, oriented perpendicular to the target nerve (FIG. 11C-4).

Blocking current was applied using a waveform that consisted of 1) a ramp-up phase from zero current, generally 2-4 seconds in duration, to 2) a plateau phase at a constant current, generally 4-10 seconds, followed by 3) ramp-down phase, generally 2-4 seconds. Ramping prevented generating action potentials in the nerve at current onset/offset. Current intensity applied ranged from 1-20 mA.

### Statistics

Blocking current was applied using a waveform that consisted of 1) a ramp-up phase from zero current, generally 2-4 seconds in duration, to 2) a plateau phase at a constant current, generally 4-10 seconds, followed by 3) ramp-down phase, generally 2-4 seconds. Ramping prevented generating action potentials in the nerve at current onset/offset. Current intensity applied ranged from 1-20 mA.

Conduction block percentage was calculated as the percent change in force output during transcutaneous direct current block as compared to pre-block baseline. Force output during tDCB was measured as the difference between peak force output and baseline during the plateau phase of tDCB, while the force output during pre-block baseline was measured as the difference between the peak force output and baseline during the five seconds preceding tDCB ramp onset. This algorithm searched for peaks at a regular frequency and if not found, for example during complete neuromotor block, noise was detected as a peak. As a result, a block of 0% was not attained. Therefore, for this study, complete neuromotor block is defined as >95% block using this analysis. Student's t-test was used to compare data in direct current and block relationship, electrode configurations and conductive gel thickness comparisons. Data were analyzed using Matlab and Microsoft Excel.

### Results

### Transcutaneous Direct Current Blockade of Motor Fiber Activity

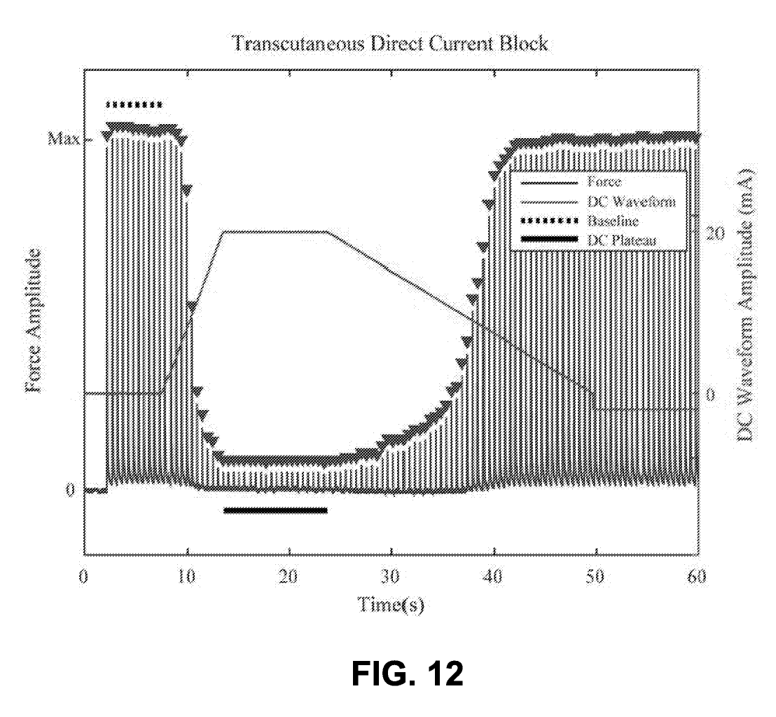

Transcutaneous direct current nerve block (tDCB) was applied to branches of the sciatic nerve in the anesthetized rat, while proximal sciatic nerve stimulation (PS) evoked leg muscle twitches as measured by a force transducer (FIG. 10). In doing so, tDCB blocked transmission between PS and distal motor output, the results of which could readily be measured by the force output. tDCB effected robust and reliable attenuation of motor output when optimal stimulation parameters and electrode geometry were used. FIG. 12 presents a typical example of tDCB providing attenuation of the PS-driven maximal motor output twitches, resulting in a stable partial block for the duration of the plateau period, or the period when the direct current was held at a constant level. Ramp-up and -down of the DC amplitude revealed a direct and dynamic relationship between the DC amplitude and level of block achieved.

### Direct Current and Block Relationship

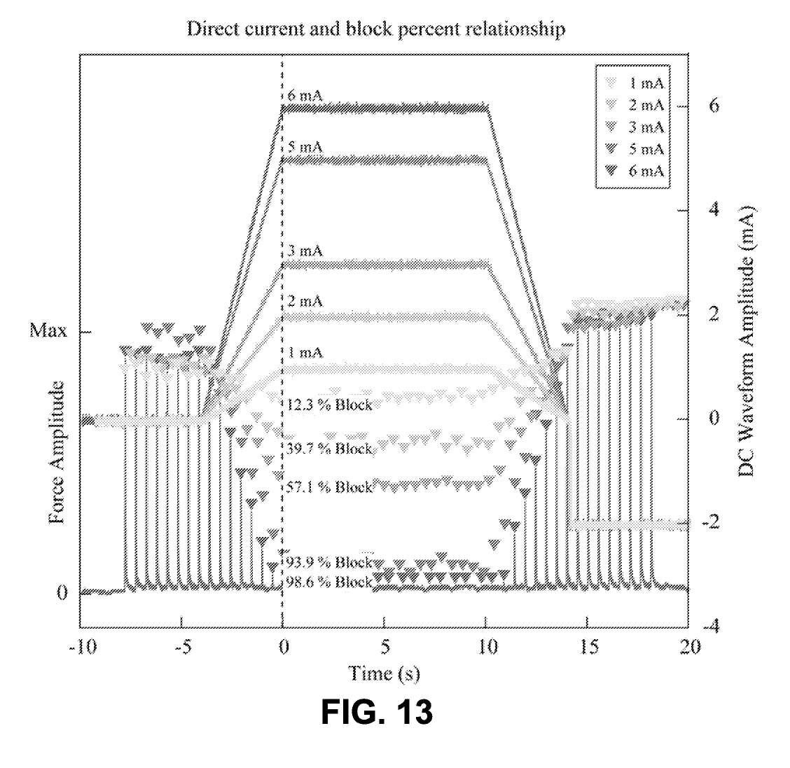

Complete or near-complete neuromotor block, manifested by elimination of PS-evoked force output, was routinely achieved at DC block amplitudes at or below 20 mA, and as low as 6 mA. A direct relationship was observed between tDCB amplitude and percent of motor block achieved. FIG. 13 provides an example of DC amplitude and block percent relationship over a number of DC amplitudes at a single site, holding all other parameters constant. As the DC blocking amplitude is increased, so too is the block percent achieved until culmination of complete block at 6 mA (>95% block; see Methods). This relationship was seen in all ten experiments, in that greater block was always achieved with greater blocking current, until complete block was achieved. We were able to induce complete to near—complete nerve conduction block in each of ten experiments. In eight out of ten experiments, complete block was achieved. In all ten experiments combined, the mean percent block in trials with the highest block percentage was 91.5%±13.0.

tDCB of Tetanic Muscle Contraction

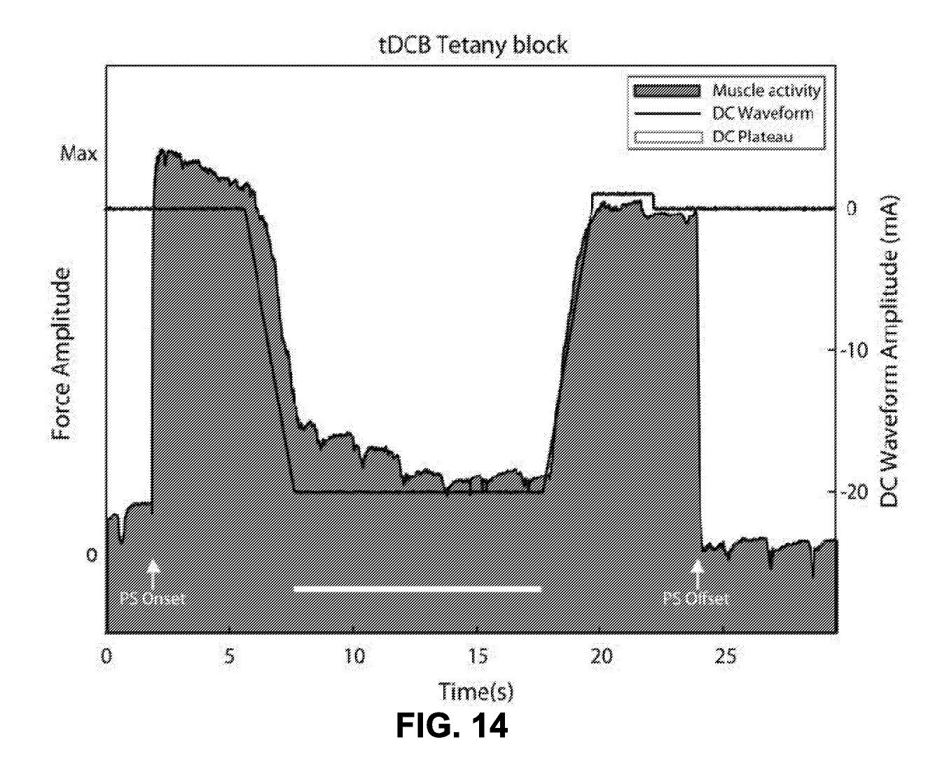

Block assessment was conducted while applying supra-maximal PS at 1-2 Hz, resulting in individual force twitches capable of being clearly delineated from baseline muscle tone. From a practical standpoint, for example in clinical cases of muscle spasticity, fused muscle output occurs resulting in tetanic muscle contraction. To determine if tDCB was capable of producing block of fused muscle activity, PS was applied at 40 Hz, resulting in tetanic muscle activity. When tDCB was applied in this setting, conduction block was achieved (FIG. 14). In this example the baseline activity modulates at −0.7 Hz, likely corresponding to respiration of the animal (˜42 breaths per minute).

### Electrode Configuration

Electrode orientation of the active and return electrodes relative to the target nerve significantly affected the ability to achieve nerve conduction block. Numerous geometric configurations were attempted (FIG. 11), with each orientation providing electrical current in a different direction relative to the target nerve. FIG. 11A illustrates hypothetical electrical field lines between the anode and cathode, with blocking success dependent upon the target nerve being within the electrical field. One electrode orientation pairing in particular had the most success in providing conduction block. Electrode geometric configuration #1 (FIG. 11B—1), with active and return electrodes placed on opposing sides and perpendicular to the leg/nerve, had the greatest blocking effect. Block was obtained sparsely and inconsistently using configuration #2 (FIG. 11B—2), in which active and return electrodes were placed on opposing sides, though not directly perpendicular, to the leg/nerve. Configurations #3 and #4 (FIG. 5C—3,4), in which the active and return electrodes were placed on the same side with respect to the leg/nerve, did not yield nerve conduction block up to current intensities of 20 mA. In a randomized data set comparing the blocking effectiveness of the four configurations, only configuration #1 had any blocking effect (23.5±7.0%), significantly greater than the other three configurations (−3.9±2.0%; p=0.001, student's t-test). The greatest block achieved in each of the 10 experiments was obtained by placing active and return electrodes in configuration #1.

### Conductive Gel Thickness Comparison

The data was obtained primarily through the use of Ag/AgCl disc electrodes (see Methods) placed medially and laterally about the nerve being blocked, with a thin layer of gel interposed between the electrodes and skin. This thin layer of gel was applied to the electrode, after which the electrode was placed on the skin and compressed with tape to secure the electrode in place, resulting in minimal conductive gel. No skin damage was observed at the end of each session when this thin layer of gel was used. Yet direct current has been known to produce apparent skin irritation in human subjects. The underlying cause of this skin irritation, primarily described as erythema, remains unclear, with factors such as heat, vasodilation and electrochemical reactions potentially being the cause. DC, when applied to the point where the total charge exceeds the electrochemical water window of the electrode interface, can result in irreversible electrochemical reactions. There are a number of ways to reduce the occurrence of these irreversible electrochemical reactions. One such method is to increase the space between the electrode surface and skin by using a greater reservoir of electroconductive gel, thereby providing an improved electrochemical buffer.

The blocking effectiveness of a thick electrode gel buffer (1 cm) placed within a rubber spacer interposed between the disc electrode and the skin was compared to that of the disc electrode with a thin layer of gel (<0.5 mm). Thirty two randomized trials between the two electrode arrangements were performed, and the block percentage was compared. The disc electrode with thin gel produced significantly greater conduction block (82.6±19.3%) than that with thick gel (63.9±14.8%; p=0.005, student's t-test). Yet these data suggest that conduction block is feasible using 1 cm of electroconductive gel, which may be preferable when applying tDCB in human subjects.

No signal attenuation indicating nerve damage was observed. No skin irritation, such as erythema, discoloration or blistering, was noted underlying the electrodes at the conclusion of each study in which conductive gel was used as an interface between the electrodes and skin. In order to assess if skin irritation would occur with no gel in place, in one trial tDCB was applied using no gel interface, so that the Ag/AgCl electrode was in direct contact with the skin. 20 mA of current was then delivered for 20 minutes. Four ˜0.5 mm red spots were observed on one ˜90° edge of the cathode electrode at the conclusion of that trial.

### Example II—Potential Clinical Applications

The tDCB described above can be used in many different clinical applications to treat a neurological disorder non-invasively by applying a DC transcutaneously to alter nerve conduction (e.g., block or attenuation). The tDCB can be reversible, so that when the tDCB is turned off, conduction can be restored in the stimulated nerve. Several non-limiting example clinical applications are described below.

### Spasticity

tDCB can be used to reduce or eliminate muscle spasticity or spasms for the purpose of preventing or reversing joint contractures. This is particularly applicable to diseases like cerebral palsy, stroke, and multiple sclerosis, as well as spinal cord injury and post-orthopedic surgery. In each of these cases, muscle spasticity and spasms can be a significant co-morbidity, causing joints to contract and remain contracted when the patient desires to relax. Over time, such contraction can lead to a physiologic shortening of the contracted muscles, causing permanent joint contractures and loss of range of motion in the joint. When these contractures occur, traditional treatments are often destructive and irreversible with often poor outcomes. For example, traditional treatment methods involve damaging nerve fibers, either chemically or surgically, or surgical sectioning of tendons. In contrast, tDCB, advantageously, can be applied to block spastic signals on motor or sensory nerves, causing the muscles to relax using a transcutaneously applied DC. In some instances, the tDCB can be applied using an open loop control system, where a patient is given a switch or other input device to turn the block on and off and to control the degree of block.

tDCB is reversible, allowing patients to relax the muscles as desired, yet reverse the block when necessary. For example, tDCB can be applied during periods of rest at night or when the individual is less active, allowing the muscles to fully relax, but shut off (reversed) when the individual is active. Since the treatment with tDCB is not destructive, so the treatment can be performed much earlier in the disease progression progress, therefore preventing contractures from occurring.

In some instances, the tDCB can be used to produce a partial nerve block, which can be beneficial with preserving motor function. In a partial block, some, but not all, of the neural signals to the muscle fibers are blocked and the muscle contraction strength is lessened. This can allow voluntary movements of the spastic muscle without triggering the overpowering contraction that is common to spastic muscles. In this case, antagonist muscles can be strong enough to move the joint through the full range of motion.

An example application of tDCB is for prevention/treatment of contractures in spastic cerebral palsy. Spastic ankle plantar flexors and hip adductors in cerebral palsy result in a characteristic pattern of contractures that limit function, make hygiene difficult and can become painful. Release of gastronomies tightness through tendon lengthening or neurolysis is usually only performed as a last resort due to the irreversible nature of these procedures. In some instances, reversible tDCB can be applied transcutaneously above the oburator nerve to relax the hip abductors and to the posterior tibial nerve to block ankle plantarflexion. The patient is able to turn off the block when movement is desired. Another examples application of tDCB is torticollis, which can be used to treat/prevent involuntary movements and spasticity that occur in conditions such as dystonias, choreas and tics by blocking the sternocleidomastoid muscle and, in some cases, block of the posterior neck muscles.

In some instances, the DC can be applied as charge balanced DC waveforms consisting of an increase to a plateau of one polarity, which can last for a time period (e.g., 10 seconds), followed by a decrease of the current to an opposite polarity, can be used for the tDCB. The plateaus of each phase can be the same, but typically the second phase is 10-30% of the amplitude of the first phase. The total charge delivery is zero or substantially less than the charge in each phase (e.g., <10% charge imbalance). The waveform produces either a depolarizing or a hyperpolarizing nerve block during the first phase plateau, and in some cases also blocks during the second phase plateau. Increasing the current from zero to the plateau is often performed slowly over the course of a few seconds in order to eliminate the generation of action potentials in the nerves. Additionally, multiple electrode contacts can be used to maintain a constant conduction block by cycling between the different contacts to deliver the DC waveform to the nerve.

### Pain

tDCB can be used to treat both acute and chronic pain due to, for example, cancer, pancreatitis, neuroma, endometriosis, post-herpetic neuralgia, back pain, headache, and joint replacement. In fact, the tDCB can be used to block any nerve conduction leading to the perception of pain as an alternative to neurolysis or chemical block. Notably, tDCB is reversible and can be used early in the treatment because if there are any side effects, they can be alleviated immediately by turning the block off. Additionally, the intensity and extent of the tDCB can be adjustable (e.g., as an open loop system).

Depending on the pain treated with the tDCB, the electrode contacts can be located near the target nerve. In some instances, the tDCB can be delivered to an autonomic nerve (e.g., the sympathetic ganglia).

In some instances, the DC can be applied as charge balanced DC waveforms consisting of an increase to a plateau of one polarity, which can last for a time period (e.g., 10 seconds), followed by a decrease of the current to an opposite polarity, can be used for the tDCB. The plateaus of each phase can be the same, but typically the second phase is 10-30% of the amplitude of the first phase. The total charge delivery is zero or substantially less than the charge in each phase (e.g., <10% charge imbalance). The waveform produces either a depolarizing or a hyperpolarizing nerve block during the first phase plateau, and in some cases also blocks during the second phase plateau. Increasing the current from zero to the plateau is often performed slowly over the course of a few seconds in order to eliminate the generation of action potentials in the nerves. Additionally, multiple electrode contacts can be used to maintain a constant conduction block by cycling between the different contacts to deliver the DC waveform to the nerve.

### Relaxation of the Urethral Sphincter

Reversible tDCB can be applied to produce a relaxation of the urinary sphincter on command (e.g., in an open loop system). An example of an application where this is important is in electrical stimulation systems designed to produce bladder evacuation for individuals with spinal cord injury. In these systems, stimulation of the sacral roots produces bladder contraction for evacuation, but also produces unwanted sphincter contraction. The methods of the present disclosure can be applied bilaterally and transcutaneously to the pudendal nerve to prevent sphincter activity during bladder activation. After the bladder is emptied, the block can be turned off to restore continence. The blocking electrode contact may also be used as stimulation to activate a weak sphincter and improve continence. Nerve conduction block on the sacral sensory roots can also be used to prevent spontaneous bladder contraction and thus improve continence. Methods can also be used to control bladder-sphincter dyssynergia in spinal cord injury.

### Hyperhidrosis

Reversible tDCB can be applied to neural structures of the sympathetic nervous system (e.g., in an open loop system) to treat hyperhidrosis (sweaty palms). The tDCB is a reversible alternative to the traditional sympathectomy, which involves a permanent surgical destruction or disruption of fibers in the sympathetic chain. Sympathectomy is permanent and may have irreversible side effects (like without an excessive reduction leading to dry skin and other side effects associated with destruction of the sympathetic system). In contrast, tDCB can accomplish the same desirable effect without producing any permanent damage to any neural structures. The tDCB can be applied transcutaneously when needed and/or adjusted so to provide a desired degree of reduction in palmar sweating, without the undesirable side effects.

In one example, the tDCB can be applied transcutaneously to specific regions of the sympathetic nervous system. For example, the tDCB can be applied transcutaneously by electrode contacts placed adjacent a targeted sympathetic ganglia so that an electric field generated with sufficient intensity so that the action potentials transmitted within or between sympathetic ganglia are blocked or down-regulated.

In some instances, the DC can be applied as charge balanced DC waveforms consisting of an increase to a plateau of one polarity, which can last for a time period (e.g., 10 seconds), followed by a decrease of the current to an opposite polarity, can be used for the tDCB. The plateaus of each phase can be the same, but typically the second phase is 10-30% of the amplitude of the first phase. The total charge delivery is zero or substantially less than the charge in each phase (e.g., <10% charge imbalance). The waveform produces either a depolarizing or a hyperpolarizing nerve block during the first phase plateau, and in some cases also blocks during the second phase plateau. Increasing the current from zero to the plateau is often performed slowly over the course of a few seconds in order to eliminate the generation of action potentials in the nerves. Additionally, multiple electrode contacts can be used to maintain a constant conduction block by cycling between the different contacts to deliver the DC waveform to the nerve.

### Sialorrhea

Sialorrhea or excessive drooling is a major issue in children with cerebral palsy and adjust with neurodegenerative disorders. Current medical management, using topical agents, oral agents, and botulinum toxim, is unsatisfactory because these treatments are ineffective or produce unwanted side effects, including lack of salivation when desired. Advantageously, tDCB can be used as an alternative to these traditional treatments that can rapidly and reversibly block activation of the salivary glands, therefore reducing saliva production when desired. The advantages of tDCB include the ability of a patient or caregiver to turn on and of the activation of the salivary glands when desired. Additionally, tDCB can provide for partial or incomplete block, reducing, but not eliminating, salivation, thereby alleviating the symptoms without producing unwanted side effects. tDCB for alleviation of sialorrhea can be applied to the nerve branches supplying the autonomic activation of the salivary glands, targeting one or more nerves. tDCB may be applied transcutaneously near each salivary gland.

In some instances, the DC can be applied as a charge balanced DC waveforms consisting of an increase to a plateau of one polarity, which can last for a time period (e.g., 10 seconds), followed by a decrease of the current to an opposite polarity, can be used for the tDCB. The plateaus of each phase can be the same, but typically the second phase is 10-30% of the amplitude of the first phase. The total charge delivery is zero or substantially less than the charge in each phase (e. g., <10% charge imbalance). The waveform produces either a depolarizing or a hyperpolarizing nerve block during the first phase plateau, and in some cases also blocks during the second phase plateau. Increasing the current from zero to the plateau is often performed slowly over the course of a few seconds in order to eliminate the generation of action potentials in the nerves. Additionally, multiple electrode contacts can be used to maintain a constant conduction block by cycling between the different contacts to deliver the DC waveform to the nerve.

From the above description, those skilled in the art will perceive improvements, changes and modifications. Such improvements, changes and modifications are within the skill of one in the art and are intended to be covered by the appended claims.

The following is claimed:

1. A method, comprising:
placing at least two electrodes on a surface of a patient's skin;
applying a direct current (DC) to a target nerve located between the at least two electrodes, wherein the DC has an amplitude sufficient to alter transmission of action potentials in the target nerve; and
altering the transmission of the action potentials in the target nerve based on an electric field generated as a result of the applied DC.

2. The method of claim 1, wherein the altering of the transmission of action potentials in the target nerve comprises blocking the transmission of the action potentials in the target nerve or attenuating the transmission of the action potentials in the target nerve.

3. The method of claim 1, wherein the at least two electrodes are geometrically arranged on the surface of the patient's skin to direct the flow of the DC in a direction sufficient to alter the transmission of the action potentials in the target nerve.

4. The method of claim 1, wherein the at least two electrodes are located on opposing sides of the target nerve.

5. The method of claim 1, wherein the DC is applied as a biphasic waveform.

6. The method of claim 5, wherein a second phase of the biphasic waveform reverses the charge delivered by a first phase of the biphasic waveform.

7. The method of claim 5, wherein a second phase of the biphasic waveform reverses less than 100% of the total charge delivered by a first phase of the biphasic waveform to reduce electrochemical reactions that are damaging to the skin surface and/or the electrodes.

8. The method of claim 5, wherein a second phase of the biphasic waveform is longer and of a lower amplitude than the first phase.

9. The method of claim 1, further comprising reverting to normal transmission of the action potentials in the target nerve within 60 seconds or less following ending the delivery of the DC.

10. The method of claim 1, further comprising altering the transmission of the action potentials in the target nerve without at least one of damaging a structure of the target nerve, damaging the skin of the patient, and producing systemic side-effects.

11. A system, comprising:
a current generator that generates a DC; and
a first skin electrode, coupled to the current generator, that delivers the DC transcutaneously through a target nerve to a second skin electrode;
wherein conduction in the target nerve is altered as a result of an electric field generated in response to the DC.

12. The system of claim 11, wherein the conduction in the target nerve is altered by blocking the transmission of action potentials in the target nerve or attenuating the transmission of action potentials in the target nerve.

13. The system of claim 11, wherein the first skin electrode and the second skin electrode are geometrically arranged on the surface of the patient's skin on opposing sides of the target nerve.

14. The system of claim 11, wherein the first skin electrode is made of an electrically-conductive material.

15. The system of claim 11, wherein the first skin electrode is coupled to the skin by a conductive electrolyte gel to improve conduction of the DC through the skin.

16. The system of claim 11, wherein the conduction of the target nerve is altered by stopping one or more action potentials from travelling through the target nerve.

17. The system of claim 11, wherein the DC is applied as a charge-balanced biphasic waveform that produces a zero net charge.

18. The system of claim 11, wherein the DC is applied as a substantially charge-balanced biphasic waveform that produces a small net charge to reduce electrochemical reactions that are damaging to the skin surface and/or the electrodes.

19. A method, comprising:
applying a DC with an amplitude sufficient to block or attenuate conduction in a target nerve via a transcutaneous electrode pair;
wherein the transcutaneous electrode pair is geometrically arranged on the surface of the patient's skin to direct the flow of the DC in a direction that facilitates generation of an electric field sufficient to block or attenuate the conduction in the target nerve.

20. The method of claim 19, wherein the conduction is reversibly blocked or attenuated in the target nerve.

21. The method of claim 19, wherein the DC is a biphasic waveform that is sufficiently charge-balanced to reduce the production of damaging electrochemical reaction products.
